(19) United States
(12) Patent Application Publication
(10) Pub. No.: US 2017/0107506 A1
(43) Pub. Date: Apr. 20, 2017

(54) SYSTEM AND METHODS FOR APPLYING CONTROLLED LOW-INTENSITY ULTRASOUND TO A CELL CULTURE

(71) Applicant: SONIFY BIOSCIENCES, LLC, Baltimore, MD (US)

(72) Inventors: Miriam Sara BOER, Baltimore, MD (US); Daniel Jordan ROGERS, Baltimore, MD (US); Soraya J. BAILEY, Baltimore, MD (US)

(73) Assignee: SONIFY BIOSCIENCES, LLC, Baltimore, MD (US)

(21) Appl. No.: 15/317,867

(22) PCT Filed: May 6, 2015
(86) PCT No.: PCT/US15/29376

§ 371 Date: Dec. 9, 2016

Related U.S. Application Data
(60) Provisional application No. 62/012,640, filed on Jun. 16, 2014.

(51) Int. Cl. C12N 13/00; C12M 1/34; C12M 1/42

(52) U.S. Cl. CPC C12N 13/00; C12M 35/04; C12M 41/12

(57) ABSTRACT

Disclosed herein are ultrasonic systems for applying low-intensity ultrasound to a sample (e.g., a cell culture or tissue). Further disclosed are methods for applying low-intensity ultrasound to a sample (e.g., a cell culture or tissue) or inducing apoptosis in a cell using the ultrasonic systems.

### CROSS-REFERENCE TO RELATED APPLICATION

This application claims benefit under 35 U.S.C. §119(e) of U.S. Provisional Application No. 62/012,640 filed Jun. 16, 2014, the contents of which are incorporated herein by reference in its entirety.

### TECHNICAL FIELD

The present invention relates to systems and methods for applying low-intensity ultrasound to cell cultures and/or tissues.

### BACKGROUND

In biology and medicine, ultrasound has been used to lyse cells, homogenize tissues and biological components, shear DNA, RNA, or chromatin, and deliver compounds into cells. Most of these applications require high-intensity ultrasound as they result in either permanent or transient mechanical damage to the cells.

Currently, no instruments exist that can deliver low-intensity ultrasound to a cell culture in a controlled, calibrated manner. The lack of such an instrument has hindered scientific research in studying the effects of low-intensity ultrasound on cells.

### SUMMARY

The present relates to a system and method for applying low-intensity ultrasound to a sample (e.g., a cell culture or tissue) in a controlled, calibrated manner.

One aspect of the invention relates to an ultrasonic system for applying low-intensity ultrasound to a cell culture, the system comprising: (i) an ultrasonic source adapted to apply low-intensity ultrasound; (ii) a bath coupled to the ultrasonic source, whereby the bath can be filled with a fluid, and whereby the ultrasonic source applies low-intensity ultrasound to the fluid; (iii) a support for supporting a cell culture container adapted to be in contact with the fluid in the bath, whereby low-intensity ultrasound is applied to the cell culture container; (iv) at least one temperature sensor configured to measure temperature of a culture medium in the cell culture container; and (v) an analysis module configured to receive information from the temperature sensor to calculate ultrasound intensity as a function of a change in the temperature of the culture medium over time.

In accordance with some embodiments of the invention, the ultrasound intensity is calculated according to a formula:

$$r = \frac{m \cdot C \cdot d T}{A \cdot d t} ,$$

where r is the ultrasound intensity, m is the mass of the culture medium, C is the specific heat capacity of the culture medium, A is the area of the culture medium in contact with a bottom surface of the cell culture container, dT is the temperature difference of the culture medium over the course of dt, and dt is time difference.

In accordance with some embodiments of the invention, the ultrasonic source comprises an ultrasonic transducer and an ultrasonic generator.

In accordance with some embodiments of the invention, the cell culture container is supported such that a bottom surface of the cell culture container is no more than 2 mm away from the ultrasonic transducer.

In accordance with some embodiments of the invention, the ultrasonic source produces ultrasound at a frequency between 20 kHz and 50 MHz.

In accordance with some embodiments of the invention, the ultrasonic source produces ultrasound at an intensity of no more than 50 W/cm².

In accordance with some embodiments of the invention, the fluid comprises water.

In accordance with some embodiments of the invention, the support comprises a positioning system that allows for changing the distance between the ultrasonic source and the cell culture container.

In accordance with some embodiments of the invention, the cell culture container is a petri dish or a well plate.

In accordance with some embodiments of the invention, the temperature sensor is a thermocouple or an infrared sensor.

In accordance with some embodiments of the invention, the system comprises two or more temperature sensors, and the analysis module averages the temperature of the culture medium based on the information provided by the temperature sensors.

In accordance with some embodiments of the invention, the system comprises at least seven thermocouples.

A related aspect of the invention regards a method of applying low-intensity ultrasound to a cell culture, the method comprising positioning a cell culture container comprising the cell culture to be in contact with the fluid in the bath of an ultrasonic system described herein and applying low-intensity ultrasound to the cell culture.

In accordance with some embodiments of the invention, the low-intensity ultrasound is applied to the cell culture with a duration of exposure of no more than 60 minutes.

In accordance with some embodiments of the invention, the cell culture comprises non-adherent cells.

In accordance with some embodiments of the invention, the cell culture comprises tumor cells, keratinocytes, osteoblasts, osteoclasts, osteocytes, chondrocytes, hepatocytes, islet cells, myocytes, epithelial cells, kidney cells, neurons, stem cells, or a combination thereof.

In accordance with some embodiments of the invention, the cell culture comprises melanoma cells.

A further aspect of the invention relates to a method of inducing apoptosis in a cell, the method comprising positioning a cell culture container comprising the cell culture to be in contact with the fluid in the bath of an ultrasonic system described herein and applying low-intensity ultrasound to the cell culture.

In accordance with some embodiments of the invention, the low-intensity ultrasound is applied to the cell culture with a duration of exposure of no more than 60 minutes.

In accordance with some embodiments of the invention, the cell culture comprises non-adherent cells.

In accordance with some embodiments of the invention, the cell culture comprises tumor cells, keratinocytes, osteoblasts, osteoclasts, osteocytes, chondrocytes, hepatocytes, islet cells, myocytes, epithelial cells, kidney cells, neurons, stem cells, or a combination thereof.

In accordance with some embodiments of the invention, the cell culture comprises melanoma cells.

### BRIEF DESCRIPTION OF THE DRAWINGS

FIG. 1 is a schematic illustration of one embodiment of the system according to the invention.

FIG. 2 is a schematic illustration of one embodiment of the system according to the invention.

FIG. 3 is a photograph of one embodiment of the ultrasonic system according to the invention.

FIG. 4 is a plot of temperature change during sonication as measured by 7 thermocouples immersed in different wells of the cell culture plate, with one channel measuring the ultrasonic bath temperature. The slopes of these lines correspond to the rate of ultrasonic energy being deposited in the wells of the culture plate.

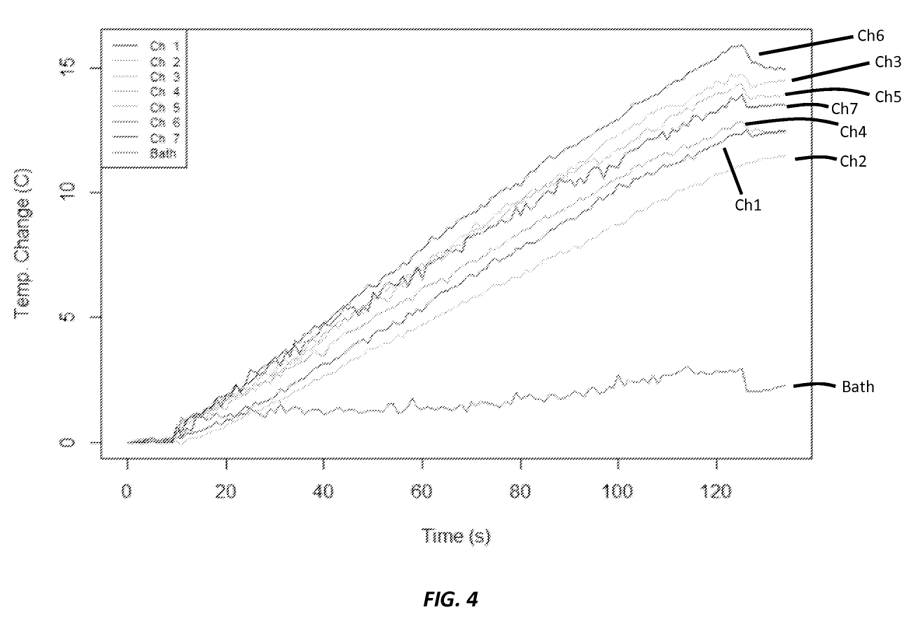

### DETAILED DESCRIPTION

Many challenges are present in designing a system that can deliver low-intensity ultrasound to a cell culture in a controlled, calibrated manner. First of all, the options of ultrasonic probes that can be used in this context are limited. When culturing cells in vitro, among the most critical of which is cleanliness of the culture environment. As such, it is impractical to expose the cells to ultrasound using a submersible ultrasonic probe due to contamination risks. Also, the radiation pattern of a submersible ultrasonic probe does not guarantee that the cells will receive uniform exposure to the ultrasonic energy. Secondly, the system must be compatible with existing cell culture plates, since the plates are specially designed for cell growth and are compatible with the rest of the laboratory infrastructure required for culturing cells in vitro. Last but not least, while a number of methods exist for calibrating ultrasound intensity in such an environment, they suffer from similar drawbacks as ultrasonic probes. Most ultrasonic power sensors, especially at higher frequencies, consist of quartz pressure sensors that measure ultrasonic pressure at a specific point location. Not only is immersing such a probe in the growth media at such small volumes impossible, but these probes also provide too spatially and temporally specific information.

Provided herein are ultrasonic systems and methods that address each and every aspect of the above-mentioned challenges.

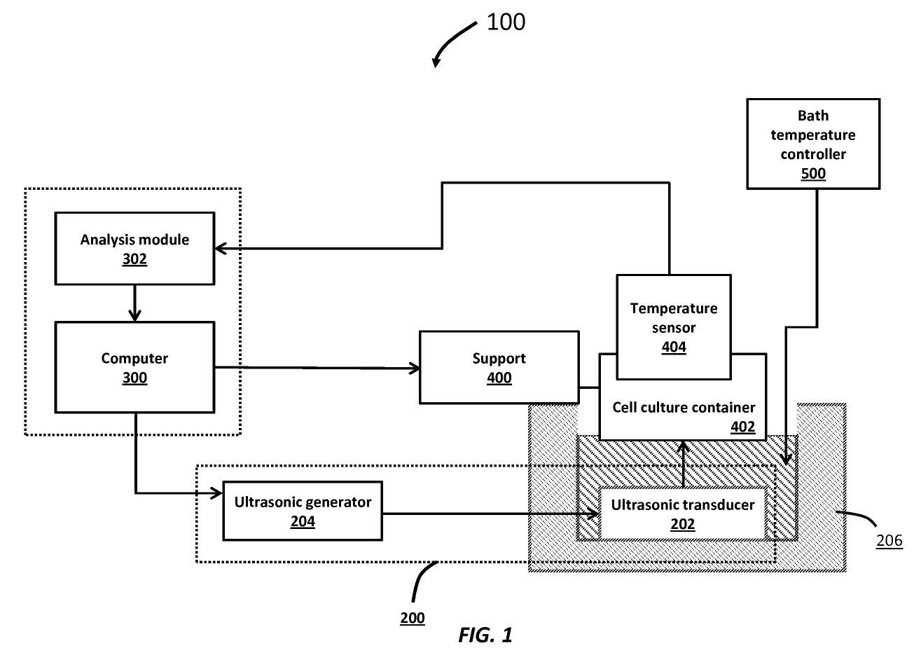

FIG. 1 is a schematic illustration of one embodiment of the system according to the invention. Specifically, FIG. 1 depicts an ultrasonic system comprising an ultrasonic source 200, a bath 206 that can be filled with a fluid such as water, a support 400 for supporting a cell culture container 402 that is in contact with the fluid, at least one temperature sensor 404 for measuring the temperature of the culture medium in the cell culture container 402, an analysis module 302 for receiving temperature information from the temperature sensor 404, a computer 300 for controlling the ultrasonic source 200 and optionally the support 400, and optionally a bath temperature controller 500 for maintaining a suitable fluid bath temperature for cell cultures.

The bath 206 should be large enough to accommodate a cell culture container 402. In accordance with some embodiments of the invention, the bath 206 can further comprise a mark on the inside wall of the bath indicating the level to which a fluid should be filled for operation. Examples of suitable cell culture containers include, but are not limited to, a petri dish and a well plate (e.g., 6, 24, 96, or 384 wells). Suitable materials for the cell culture containers include, but are not limited to, plastic such as polystyrene, or glass. The cell culture container 402 can serve as a substrate for a sample (e.g., a cell culture or tissue) that will receive an ultrasound treatment. The bath 206 can comprise materials such as metal, ceramics, or plastics.

In accordance with some embodiments of the invention, the ultrasonic source 200 can comprise an ultrasonic transducer 202 and an ultrasonic generator 204. The ultrasonic generator 204 can be configured to provide input to the ultrasonic transducer 202 and drive the generation of low-intensity ultrasound signals. In accordance with some embodiments of the invention, the ultrasonic transducer 202 can be positioned on the bottom of the bath 206. A low-intensity ultrasound generated by the ultrasonic transducer 202 can propagate through the fluid and impinge upon the cell culture container 402. The low-intensity ultrasound can further propagate through the cell culture container 402 and impinge on the sample in the container.

In accordance with some embodiments of the invention, the support 400 can be a scaffold, a rig, or a bracing system for suspending and securing the cell culture container 402. The cell culture container 402 can be loaded into a sample holder of the support 400, and then optionally secured by means such as pins, screws, or clamps. In accordance with some embodiments of the invention, the sample holder can be fixed at a pre-determined distance relative to the ultrasonic transducer 202. The pre-determined distance can be the distance at which the cell culture container 402 can receive optimal ultrasound intensity. In accordance with some embodiments of the invention, the pre-determined distance is between 1 mm and 20 mm, between 1 mm and 10 mm, or between 1 mm and 5 mm. In accordance with some embodiments of the invention, the pre-determined distance is about 2 mm.

In accordance with some embodiments of the invention, the support 400 can comprise a positioning system. The positioning system can allow the cell culture container 402, once loaded, to move into the proper position relative to the ultrasonic transducer 202. In accordance with some embodiments of the invention, the positioning system, controlled by the computer 300, can include at least one motorized linear stage that permits the cell culture container 402 to be moved according to a Cartesian coordinate system. The positioning system can move the cell culture container 402 relative to the ultrasonic transducer 202 in one, two, or three dimensions (i.e., x, y, and z). The positioning system can be controlled by the computer 300 through commercially available motion control board and stepper motor power amplifier device.

At least 1 (e.g., 1, 2, 3, 4, 5, 6, 7, 8, or more) temperature sensor 404 are used to measure the temperature of the culture medium in the cell culture container 402 when ultrasound is applied. If the cell culture container 402 includes 2 or more wells (e.g., 2, 3, 4, 5, 6, 7, 8, 9, or more), the temperature sensors can be distributed in a number of the wells so that an average temperature can be obtained. The average temperature can be the numerical average of all or some of the temperatures acquired by the temperature sensors. In accordance with some embodiments of the invention, the temperature sensor 404 can be a contact-type temperature sensor (e.g., a thermistor or a thermocouple). In accordance with some embodiments of the invention, the temperature sensor can be a non-contact temperature sensor such as an infrared sensor.

An analysis module 302 is configured to receive temperature information from the temperature sensor 404. The analysis module 302 can calculate ultrasound intensity as a function of a change in the temperature of the culture medium over time. The ultrasound treatment can heat up the culture medium in at least the following two ways: the culture medium absorbs acoustic energy and coverts it to heat; the cell culture container absorbs acoustic energy and converts it to heat which, in turn can heat the culture medium. The higher the ultrasound intensity, the faster the culture medium gets heated up. Therefore how fast the temperature changes can be a good indication of the ultrasound intensity. For example, the ultrasound intensity can be calculated according to a formula:

$$r = \frac{m \cdot C \cdot d T}{A \cdot d t} ,$$

where r is the ultrasound intensity, m is the mass of the culture medium, C is the specific heat capacity of the culture medium, A is the area of the culture medium in contact with a bottom surface of the cell culture container, dT is the temperature difference of the culture medium over the course of dt, and dt is time difference. The specific heat capacity of the culture medium can be approximated by the specific heat capacity of water (C_{water}=4.18 J/(gK)), but can vary depending on the contents and the concentration of the contents. Methods for determining the specific heat capacity of a fluid are well known in the art and are not discussed in detail herein.

The analysis module 302 permits a user to determine the ultrasound intensity at which a sample is exposed to and monitor it in real time. The ultrasound is thus applied in a controlled and calibrated manner. By “controlled and calibrated manner” it is meant that ultrasound intensity may be adjusted and set as needed for a particular sample.

The analysis module 302 can be a stand-alone unit or can be part of the computer 300. It should be noted that other methods of measuring ultrasound intensity can be employed, such as the use of a hydrophone.

The computer 300 can include one or more programs (e.g., 1, 2, 3, 4, 5, 6, 7, 8, or more) for controlling the ultrasonic generator 204 and the positioning system, and thus the computer 300 can control the application of low-intensity ultrasound to the sample. In accordance with some embodiments of the invention, the computer 300 can further comprise a display unit for displaying information such as the temperature information and calculated ultrasound intensity. In accordance with some embodiments of the invention, one or more of the programs can be automated. For example, when a program for controlling the positioning system is automated, a user can load a cell culture container and initiate an automated positioning procedure. An automated positioning system can permit consistent positioning of cell culture containers. In another example, when a program for controlling the ultrasonic generator is automated, a user can initiate an automated ultrasound treatment session after a cell culture container is positioned properly. It should be noted that one program can be used to control both the positioning system and the ultrasonic generator.

In accordance with some embodiments of the invention, one or more of the programs can be configured to receive input from a user, for example, through a user interface. For instance, a user can specify the ultrasound intensity to be applied, the duration of exposure, the frequency of the ultrasound, or a combination thereof. The computer 300 can allow a user to store that information as a recipe so that the recipe can be run in the future without the user having to enter the same input again. By way of another example, a user can also provide input to the computer to specify the position of the cell culture container 402 (e.g., the distance between the cell culture container 402 and the ultrasonic transducer 202).

In accordance with some embodiments of the invention, the computer 300 can further include a feedback loop to automatically modify the ultrasound treatment session. For example, based on the temperature information, the computer 300 may increase or decrease the ultrasound intensity, or lengthen or shorten the duration of exposure. In a more specific example, if the temperature of the culture medium deviates excessively during treatment from a set-point temperature due to absorbed acoustic energy, the computer 300 may proportionally shorten the duration of exposure. In another example, if the calculated ultrasound intensity is not at the desired intensity set-point, the computer 300 may increase or decrease the ultrasound intensity until such set-point is reached.

The bath temperature controller 500 can comprise a temperature sensor, a heating element, and/or a cooling element for maintaining a suitable fluid bath temperature for cells. The bath temperature controller 500 may further comprise a stirring system for stirring the fluid in the bath. The optimal temperature for cell culture can depend on factors such as the body temperature of the host from which the cells were isolated and the anatomical variation in temperature. It is also known that for cell culture, overheating is a more serious problem than underheating. Most human and mammalian cell lines are maintained at 36° C. to 37° C. for optimal growth.

Various aspects of the embodiment of FIG. 1 and of components of the embodiment shown in FIG. 1, as well as other embodiments with the same, similar, and/or different components, are described in more detail below.

### Ultrasonic Source

Technologies for producing ultrasound are well known in the art. Generally, an ultrasonic transducer converts one form of energy to ultrasonic energy. In accordance with some embodiments of the invention, the ultrasonic transducer comprises 1, 2, 3, 4, 5, 6, 7, 8, 9, 10, or more piezoelectric elements, and each piezoelectric element can convert electrical energy to ultrasound. The piezoelectric elements can be arranged in a pre-defined, irregular or regular pattern (e.g., rectangular, circular, octagonal, hexagonal, etc.). The piezoelectric element comprises a piezoelectric material that includes, but is not limited to, quartz, gallium orthophosphate, langasite, lead zirconate titanate, lead titanate, lead metaniobate, barium titanate, potassium niobate, lithium niobate, lithium tantalite, sodium tungstate, zinc oxide, Ba₂NaNb₅O₅, Pb₂KNb₅O₁₅, sodium potassium niobate, bismuth ferrite, sodium niobate, bismuth titanate, sodium bismuth titanate, polyvinylidene fluoride, or any combination thereof. In some embodiments, the ultrasonic transducer comprises a magnetostrictive material, and the magnetostrictive material can produce ultrasound when exposed to a magnetic field. Magnetostrictive materials include, but are not limited to, nickel, Fe—Al alloy, Fe—Ni alloy, Co—Ni alloy, Fe—Co alloy, Co—Fe—V alloy, CoFe₂O₄, NiFe₂O₄, or any combination thereof. The ultrasonic transducer can further comprise components such as electrodes, backing, and wear plate. These components are common in ultrasonic transducers and should be apparent to those skilled in the art.

The ultrasonic source can produce ultrasound having frequency in the range of 20 kHz to 100 MHz, 20 kHz to 50 MHz, 20 kHz to 25 MHz, 20 kHz to 10 MHz, 20 kHz to 5 MHz, 20 kHz to 2.5 MHz, or 27 kHz to 2.2 MHz. In accordance with some embodiments of the invention, the ultrasound frequency is about 27 kHz. In accordance with some embodiments of the invention, the ultrasound frequency is about 2.2 MHz. In accordance with some embodiments of the invention, the ultrasound frequency is not tunable. In accordance with some embodiments of the invention, the ultrasound frequency can be tuned. The ultrasound intensity at the focal zone can be changed, for example, by varying the magnitude of the voltage applied to the piezoelectric element. The ultrasound intensity at the focal zone is no more than 75 W/cm², no more than 50 W/cm², no more than 40 W/cm², no more than 25 W/cm², no more than 10 W/cm², no more than 5 W/cm², no more than 2 W/cm², no more than 1 W/cm², or no more than 0.5 W/cm². The ultrasonic source can produce continuous or pulsed ultrasound.

A generally vertically oriented focused ultrasound beam may be generated in several ways. For example, a single-element piezoelectric transducer, such as those supplied by Sonic Concepts, Woodinville, Wash., that can be a 1.1 MHz focused single-element transducer, can have a spherical transmitting surface that is oriented such that the focal axis is vertical. Another embodiment uses a flat unfocused transducer and an acoustic lens to focus the beam. Still another embodiment uses a multi-element transducer such as an annular array in conjunction with focusing electronics to create the focused beam. The annular array potentially can reduce acoustic sidelobes near the focal point by means of electronic apodizing, that is by reducing the acoustic energy intensity, either electronically or mechanically, at the periphery of the transducer. This result can be achieved mectically by partially blocking the sound around the edges of a transducer or by reducing the power to the outside elements of a multi-element transducer. This reduces sidelobes near the energy focus, and can be useful to reduce heating of the vessel. Alternatively, an array of small transducers can be synchronized to create a converging beam. Yet still another embodiment combines an unfocused transducer with a focusing acoustic mirror to create the focused beam. This embodiment can be advantageous at lower frequencies when the wavelengths are large relative to the size of the transducer. The axis of the transducer of this embodiment can be horizontal and a shaped acoustic mirror used to reflect the acoustic energy vertically and focus the energy into a converging beam.

Heating of the cell culture container can be reduced by minimizing acoustic sidelobes near the focal zone. Sidelobes are regions of high acoustic intensity around the focal point formed by constructive interference of consecutive wavefronts. The sidelobes can be reduced by apodizing the transducer either electronically, by operating the outer elements of a multi-element transducer at a lower power, or mechanically, by partially blocking the acoustic waves around the periphery of a single element transducer. Sidelobes may also be reduced by using short bursts, for example in the range of about 3 to about 5 cycles in the treatment protocol.

The ultrasonic generator can be connected to the ultrasonic transducer either with or without a cable. In accordance with some embodiments of the invention, the ultrasonic generator can comprise a waveform generator and a RF amplifier. The ultrasonic generator can generate a variety of useful alternating voltage waveforms to drive the ultrasonic source. An operator can define the parameters of the waveforms (e.g., number of waveforms, amplitude, or frequency), or select a program defining the same. The waveform of focused ultrasound can be a single wave pulse, a series of individual wave pulses, a series of wave bursts of several cycles each, or a continuous waveform. Incident waveforms can be focused directly by either a single element, such as a focused ceramic piezoelectric ultrasonic transducer, or by an array of elements with their paths converging to a focus. Alternatively, multiple foci can be produced to provide ultrasonic treatment to multiple treatment zones, cell culture containers, or wells.

Reflected waveforms can be focused with a parabolic reflector, such as is used in an “electromagnetic” or spark-gap type shock-wave generator. Incident and reflected waveforms can be directed and focused with an ellipsoidal reflector such as is used in an electrohydraulic generator. Waveforms can also be channeled.

Technologies for waveform generators are well known in the art. Exemplary waveform generators for ultrasound can also be found in U.S. Pat. No. 3,967,143, U.S. Pat. No. 6,122,223, US20070249969, US20110215673, and US20130197401, the contents of each of which are incorporated by reference in its entirety.

### Positioning System

As used herein, x and y axes define a plane that is substantially horizontal relative to ground and/or a base of an ultrasonic system of the invention, while the z axis lies in a plane that is substantially vertical relative to the ground and/or the base of the ultrasonic system and perpendicular to the x-y plane.

In accordance with some embodiments of the invention, the positioning system can be coupled with drive electronics and devices for positioning of the cell culture container. In accordance with some embodiments of the invention, the positioning sequence can be pre-programmed, for example, in a computer, and can be executed automatically when a cell culture container is loaded onto the ultrasonic system. The drive electronics can include a waveform generator matching network, a radio frequency (RF) switch or relay, and a RF amplifier, for safety shutdown.

In accordance with some embodiments of the invention, the positioning system can comprise a three axis Cartesian positioning and motion control system to position the cell culture container. Alternative configurations may employ a combination of linear and rotary motion control elements to achieve the same capabilities as the three axis Cartesian system. Alternative positioning systems may be constructed of self-contained motor-driven linear or rotary motion elements mounted to each other and to a base plate to achieve two- or three-dimensional motion.

In accordance with some embodiments of the invention, the positioning system comprises at least one stepper motor. The stepper motor can move along at least one axis (e.g., x, y, or z). Stepper motors drive linear motion elements through lead screws to position the cell culture container. The stepper motors can be driven and controlled by means of a program (e.g., a LabVIEW software) controlling a stepper motor control board. The output signals from the control board can be amplified by a multi-axis power amplifier interface.

In accordance with some embodiments of the invention, the cell culture container can be moved in relative to the ultrasonic transducer and the other parts of the system. In accordance with some embodiments of the invention, the ultrasonic transducer can be moved while the sample holder remains fixed, relative to the other parts of the system.

The positioning system, when used in conjunction with the analysis module, can permit automated adjustment of the ultrasound intensity that is applied to the sample. Instead of adjusting the ultrasound intensity at the focal zone, the positioning system can move the sample relative to the ultrasound transducer until the sample can be exposed to the desired ultrasound intensity.

### Applications

The ultrasonic systems described herein are particularly useful in studying the effects of low-intensity ultrasound on cells in a cell culture or a tissue. These studies might in turn lead to the use of low-intensity ultrasound in, for example, therapeutic applications. For example, without limitation, one skilled in the art might be interested in using the ultrasound systems described herein to study whether low-intensity ultrasound can stimulate cell growth, or whether low-intensity ultrasound can stimulate or reduce certain cellular activity, or whether low-intensity ultrasound can reduce cell viability, or whether low-intensity ultrasound can stimulate stem cell differentiation.

In one aspect of the invention, the ultrasonic systems described herein can be used to apply low-intensity ultrasound to a sample (e.g., a cell culture or tissue), the method comprising positioning a cell culture container comprising the sample to be in contact with the fluid in the bath of an ultrasonic system described herein and applying low-intensity ultrasound to the sample.

The sample can also be a 2D or 3D scaffold comprising cells, or an engineered tissue construct. An engineered tissue construct is a three dimensional mass of living mammalian tissue produced primarily by growth in vitro that shares critical structural and functional characteristics with intact tissue, such as distinctive multicellular organization and oriented contractile function. Engineered tissue constructs include, but are not limited to, a muscular construct, a vascular construct, an esophageal construct, an intestinal construct, a rectal construct, an ureteral construct, a cartilaginous construct, a cardiac construct, a liver construct, a bladder construct, a kidney construct, a pancreatic construct, a skeletal construct, a filamentous/ligament construct, a lung construct, a neural construct, a bone construct, and a skin construct.

The low-intensity ultrasound described herein is not limited to any particular cells or tissues. Simply by way of example, suitable cells include, but are not limited to, tumor cells (e.g., melanoma cells), skin cells, osteoblasts, osteoclasts, osteocytes, chondrocytes, hepatocytes, islet cells, myocytes, epithelial cells, kidney cells, neurons, and stem cells (e.g., embryonic stem cells (ESCs) or induced pluripotent stem cells (iPSCs)), while suitable tissues include, without limitation, skin, brain, bone, cartilage, liver, pancreas, muscle, epithelium, kidney, uterus, ovarian, breast, and testes.

In accordance with some embodiments of the invention, the cell culture comprises adherent cells. In accordance with some embodiments of the invention, the cell culture comprises non-adherent cells (i.e., suspension cells). The cells are cultured or maintained using standard cell culture procedures.

A user can determine the duration of exposure for each ultrasound treatment. In accordance with some embodiments of the invention, the duration of exposure is no more than 90 minutes, no more than 70 minutes, no more than 60 minutes, no more than 50 minutes, no more than 40 minutes, no more than 30 minutes, no more than 20 minutes, or no more than 10 minutes. In accordance with some embodiments of the invention, the duration of exposure is about 0.5 minute to 60 minutes (e.g., about 0.5 minute to about 5 minutes, about 0.5 minute to about 10 minutes, about 0.5 minute to about 20 minutes, about 0.5 minute to about 30 minutes, about 0.5 minute to about 40 minutes, about 0.5 minute to about 50 minutes, about 2 minutes to about 20 minutes, about 2 minutes to about 40 minutes, about 5 minutes to about 20 minutes, about 5 minutes to about 40 minutes, about 10 minutes to about 60 minutes, or about 10 minutes to about 40 minutes).

A user can also determine the ultrasound exposure frequency for a particular sample. For example, the exposure frequency can be a few times per day (e.g., once per day, twice per day, three times per day), once every two days, once every three days, once every four days, once every five days, once every six days, once every week, once every two weeks, etc.

It should be noted that the parameters of ultrasound treatment will vary depending on factors such as the cell type and desired results, and should be optimized for each cell culture or tissue to produce optimal results.

In another aspect, the ultrasonic systems described herein can be used to induce apoptosis to a cell in a sample (e.g., a cell culture or tissue) by applying low-intensity ultrasound to the cell. This can be particularly useful in cancer treatment. In accordance with some embodiments of the invention, the methods described herein can be used to determine whether two or more cell types respond differently to low-intensity ultrasound. The difference in response can be exploited to using low-intensity ultrasound in differential treatment.

In accordance with some embodiments of the invention, at least 5%, at least 10%, at least 20%, at least 40%, at least 60%, or at least 80% more of the cells exposed to low-intensity ultrasound die through apoptosis than the amount of apoptosis observed in a control sample without low-intensity ultrasound treatment.

Apoptosis can be measured by detecting the presence or absence of fragmented DNA in a sample of nucleic acids from the cell. In accordance with some embodiments of the invention, the methods described herein can be used to determine whether a certain cell type has an increased or decreased likelihood of apoptosis due to its exposure to low-intensity ultrasound. Apoptosis can also be measured by observing morphological features of the cell, by measuring the amount of histone complexes comprising fragmented DNA in a sample, by measuring the activity or determining the presence of proteases characteristically involved in apoptotic processes (e.g., such as caspase 3). Apoptosis can also be assayed by measuring membrane alterations, including: loss of terminal sialic acid residues from the side chains of cell surface glycoproteins, exposure of new sugar residues; emergence of surface glycoproteins that may serve as receptors for macrophage-secreted adhesive molecules such as thrombospondin; and loss of asymmetry in cell membrane phospholipids, altering both the hydrophobicity and charge of the membrane surface.

In particular, the human anticoagulant annexin V is a 35-36 kilodalton, Ca²⁺-dependent phospholipid-binding protein that has a high affinity for phosphatidylserine (PS). In normal viable cells, PS is located on the cytoplasmic surface of the cell membrane. However, in apoptotic cells, PS is translocated from the inner to the outer leaflet of the plasma membrane, thus exposing PS to the external cellular environment. Annexin V may therefore be used to detect phosphatidylserine asymmetrically exposed on the surface of apoptotic cells (Homburg et al., 1995, Blood 85: 532; Verhoven et al., 1995, J. Exp. Med. 182: 1597).

DNA stains such as DAPI, ethidium bromide and propidium iodide, and the like, also may be used for differential staining to distinguish viable and non-viable cells. Profiles of DNA content additionally may be used since permeabilized apoptotic cells leak low molecular weight DNA. In one aspect, detection of “sub-G 1 peaks”, or “A 0” cells (cells with lower DNA staining than that of G 1 cells) by flow cytometry is used to identify apoptotic cells in a sample. Morphological changes characteristic of apoptosis also may be detected in this manner.

Methods for studying apoptosis in individual cells are also available, such as ISNT and TUNEL enzymatic labeling assays which are known in the art. Extensive DNA degradation is a characteristic event which often occurs during the early stages of apoptosis. Cleavage of DNA yields double-stranded, low molecular weight DNA fragments (mono- and oligonucleosomes) as well as single strand breaks (“nicks”) in high molecular weight-DNA. In TUNEL, such DNA strand breaks are detected by enzymatic labeling of the free 3′-OH termini with suitable modified nucleotides (such as X-dUTP, X=biotin, DIG or fluorescein). Suitable labeling enzymes include DNA polymerases (nick translation polymerases) in ISNT, e.g., where “in situ nick translation” is performed, and terminal deoxynucleotidyl transferase in TUNEL, e.g., where end labeling is performed (see, e.g., Huang, P. & Plunkett, W., 1992, Anal. Biochem. 207: 163; Bortner et al., 1995, Trends Cell Biol. 5: 21).

Detection of apoptosis-related proteins such as ced-3, ced-4, ced-9 (Ellis and Horvitz, 1986, Cell 44: 817-829; Yuan and Horvitz, 1990, Dev. Biol. 138: 33-41; Hentgartner et al., 1992, Nature 356: 494-499.); Fas (CD95/Apo-1; Enari et al., 1996, Nature 380: 723-726), Bcl-2 (Baffy et al., 1993, J. Biol. Chem. 268: 6511-6519; Miyashita and Reed, 1993, Blood 81: 151-157; Oltvai et al., 1993, Cell 74: 609-619); p53 (Yonish-Rouach, et al., 1991, Nature 352: 345-347), e.g., such as by the use of antibodies, also may be used to assay apoptosis.

Other applications are also contemplated, for example, introduction of a material into a cell. For instance, the material is suspended in the culture medium where the cells are. By applying low-intensity ultrasound to the cells, the material can be transported into the cytoplasm of the cells.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although any known methods, devices, and materials may be used in the practice or testing of the invention, the methods, devices, and materials in this regard are described herein.

Some embodiments of the invention are listed in the following numbered paragraphs:

1. An ultrasonic system for applying low-intensity ultrasound to a cell culture, the system comprising:
(i) an ultrasonic source adapted to apply low-intensity ultrasound;
(ii) a bath coupled to the ultrasonic source, whereby the bath can be filled with a fluid, and whereby the ultrasonic source applies low-intensity ultrasound to the fluid;
(iii) a support for supporting a cell culture container adapted to be in contact with the fluid in the bath, whereby low-intensity ultrasound is applied to the cell culture container;
(iv) at least one temperature sensor configured to measure temperature of a culture medium in the cell culture container; and
(v) an analysis module configured to receive information from the temperature sensor to calculate ultrasound intensity as a function of a change in the temperature of the culture medium over time.
2. The ultrasonic system of paragraph 1, wherein the ultrasound intensity is calculated according to a formula:

$$r = \frac{m \cdot C \cdot d T}{A \cdot d t}$$

wherein r is the ultrasound intensity, m is the mass of the culture medium, C is the specific heat capacity of the culture medium, A is the area of the culture medium in contact with a bottom surface of the cell culture container, dT is the temperature difference of the culture medium over the course of dt, and dt is time difference.
3. The ultrasonic system of paragraph 1, wherein the ultrasonic source comprises an ultrasonic transducer and an ultrasonic generator.
4. The ultrasonic system of paragraph 3, wherein the cell culture container is supported such that a bottom surface of the cell culture container is no more than 2 mm away from the ultrasonic transducer.
5. The ultrasonic system of paragraph 1, wherein the ultrasonic source produces ultrasound at a frequency between 20 kHz and 50 MHz.
6. The ultrasonic system of paragraph 1, wherein the ultrasonic source produces ultrasound at an intensity of no more than 50 W/cm2.
7. The ultrasonic system of paragraph 1, wherein the fluid comprises water.
8. The ultrasonic system of paragraph 1, wherein the support comprises a positioning system that allows for changing the distance between the ultrasonic source and the cell culture container.
9. The ultrasonic system of paragraph 1, wherein the cell culture container is a petri dish or a well plate.
10. The ultrasonic system of paragraph 1, wherein the temperature sensor is a thermocouple or an infrared sensor.
11. The ultrasonic system of paragraph 1, wherein the system comprises two or more temperature sensors, whereby the analysis module averages the temperature of the culture medium based on the information provided by the temperature sensors.
12. The ultrasonic system of paragraph 11, wherein the system comprises at least seven thermocouples.
13. A method of applying low-intensity ultrasound to a cell culture, the method comprising
(i) positioning a cell culture container comprising the cell culture to be in contact with the fluid in the bath of an ultrasonic system of any one of paragraphs 1-12; and
(ii) applying low-intensity ultrasound to the cell culture.
14. The method of paragraph 13, wherein the low-intensity ultrasound is applied to the cell culture with a duration of exposure of no more than 60 minutes.
15. The method of paragraph 13, wherein the cell culture comprises non-adherent cells.
16. The method of paragraph 13, wherein the cell culture comprises tumor cells, keratinocytes, osteoblasts, osteoclasts, osteocytes, chondrocytes, hepatocytes, islet cells, myocytes, epithelial cells, kidney cells, neurons, stem cells, or a combination thereof.
17. The method of paragraph 16, wherein the cell culture comprises melanoma cells.
18. A method of inducing apoptosis in a cell, the method comprising
(i) positioning a cell culture container comprising the cell to be in contact with the fluid in the bath of an ultrasonic system of any one of paragraphs 1-12; and
(ii) applying low-intensity ultrasound to the cell culture.
19. The method of paragraph 18, wherein the low-intensity ultrasound is applied to the cell with a duration of exposure of no more than 60 minutes.
20. The method of paragraph 18, wherein the cell is non-adherent.
21. The method of paragraph 18, wherein the cell is a tumor cell, a keratinocyte, an osteoblast, an osteoclast, an osteocyte, a chondrocyte, a hepatocyte, an islet cell, a myocyte, an epithelial cell, a kidney cell, a neuron, or a stem cell.
22. The method of paragraph 21, wherein the tumor cell is a melanoma cell.

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

As used herein, the term “low intensity ultrasound” refers to ultrasound having frequency of 20 kHz or higher and an intensity at the focal zone of no more than 75 W/cm².

“Focal zone” or “focal point” as used herein means an area where ultrasound converges and/or impinges on a target, although that area of convergence is not necessarily a single focused point.

As used herein, the term “well plate” and other grammatical forms of this term can mean a plate that includes one or more wells into which samples may be deposited.

As used herein, the term “apoptosis” (“normal” or “programmed” cell death) refers to the physiological process by which unwanted or useless cells are eliminated during development and other normal biological processes. Apoptosis is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine dependent tissue atrophy. Cells undergoing apoptosis show characteristic morphological and biochemical features. These features include chromatin aggregation, nuclear and cytoplasmic condensation, partition of cytoplasm and nucleus into membrane bound vesicles (apoptotic bodies) which contain ribosomes, morphologically intact mitochondria and nuclear material. In vivo, these apoptotic bodies are rapidly recognized and phagocytized by either macrophages or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis”.

Other than in the operating examples, or where otherwise indicated, all numbers expressing quantities of ingredients or reaction conditions used herein should be understood as modified in all instances by the term “about.” The term “about” when used in connection with percentages may mean±1% of the value being referred to. For example, about 100 means from 99 to 101.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example: Some Examples of the Ultrasonic System According to the Invention

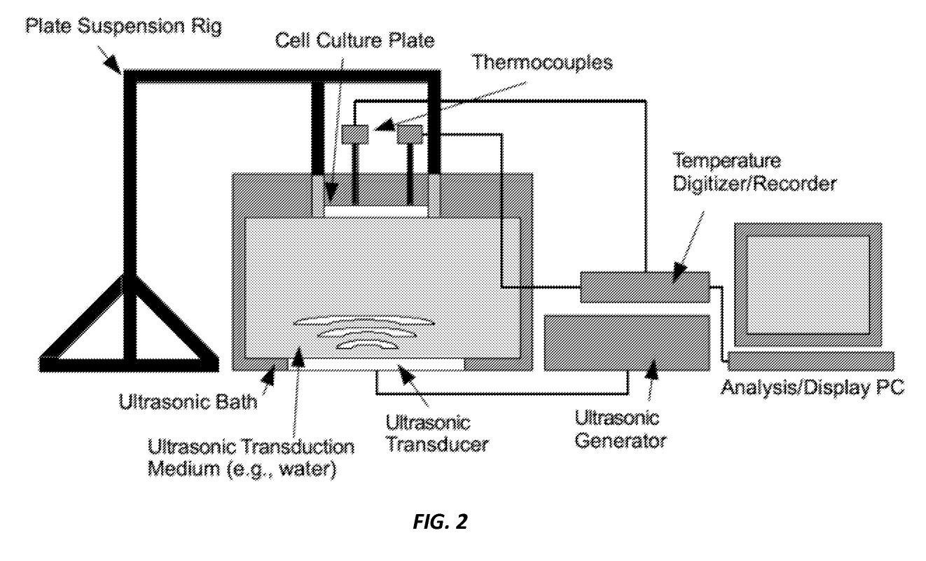

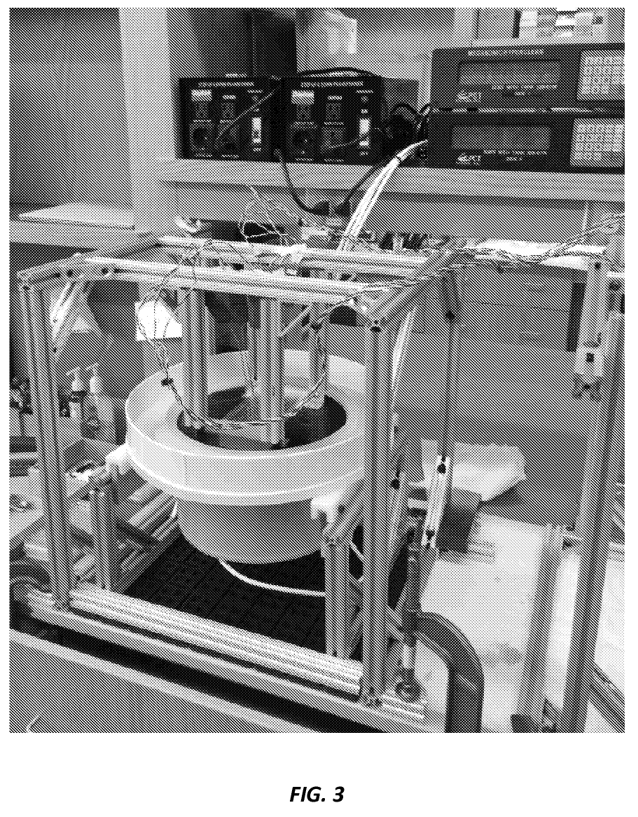

FIG. 2 is a schematic illustration of one embodiment of the system according to the invention. FIG. 3 is a photograph of one embodiment of the ultrasonic system according to the invention. In FIGS. 2 and 3, the cell culture plate is suspended via a mechanical bracing system over an ultrasonic bath, which consists of one or more ultrasonic transducers mounted to a vessel containing water or some other transduction medium that can be temperature controlled. The plate is suspended and fixed in place such that its bottom facet is just immersed in the liquid medium. The transducers then induce ultrasonic vibrations in the liquid, which disperses them and transfers them to the cell plate, which finally transfers them to the cells and cellular media within the plate wells.

Furthermore, the system comprises a bolometric method to monitor and control the level of ultrasonic energy introduced to the cells and cellular media by measuring a change in temperature over time of the volume within the plate wells and calculating the energy dispersed within those wells. Electrical thermocouples are immersed in seven equally spaced plate wells filled with media, and their output is digitally recorded over a duration of time by a computer. The slope of the linear increase in temperature as the plate is exposed to the ultrasound is measured and converted to power units using the following formula:

$$r = \frac{m \cdot C \cdot d T}{A \cdot d t} ,$$

where r is the ultrasound intensity, m is the mass of the culture medium, C is the specific heat capacity of the culture medium, A is the area of the culture medium in contact with the bottom surface of the cell culture container, dT is the temperature difference of the culture medium over the course of dt, and dt is time difference.

What is claimed is:

1. An ultrasonic system for applying low-intensity ultrasound to a cell culture, the system comprising:
(i) an ultrasonic source adapted to apply low-intensity ultrasound;
(ii) a bath coupled to the ultrasonic source, whereby the bath can be filled with a fluid, and whereby the ultrasonic source applies low-intensity ultrasound to the fluid;
(iii) a support for supporting a cell culture container adapted to be in contact with the fluid in the bath, whereby low-intensity ultrasound is applied to the cell culture container;
(iv) at least one temperature sensor configured to measure temperature of a culture medium in the cell culture container; and
(v) an analysis module configured to receive information from the temperature sensor to calculate ultrasound intensity as a function of a change in the temperature of the culture medium over time.

2. The ultrasonic system of claim 1, wherein the ultrasound intensity is calculated according to a formula:
$$r = \frac{m \cdot C \cdot d T}{A \cdot d t} ,$$
wherein r is the ultrasound intensity, m is the mass of the culture medium, C is the specific heat capacity of the culture medium, A is the area of the culture medium in contact with a bottom surface of the cell culture container, dT is the temperature difference of the culture medium over the course of dt, and dt is time difference.

3. The ultrasonic system of claim 1, wherein the ultrasonic source comprises an ultrasonic transducer and an ultrasonic generator.

4. The ultrasonic system of claim 3, wherein the cell culture container is supported such that a bottom surface of the cell culture container is no more than 2 mm away from the ultrasonic transducer.

5. The ultrasonic system of claim 1, wherein the ultrasonic source produces ultrasound at a frequency between 20 kHz and 50 MHz.

6. The ultrasonic system of claim 1, wherein the ultrasonic source produces ultrasound at an intensity of no more than 50 W/cm².

7. The ultrasonic system of claim 1, wherein the fluid comprises water.

8. The ultrasonic system of claim 1, wherein the support comprises a positioning system that allows for changing the distance between the ultrasonic source and the cell culture container.

9. The ultrasonic system of claim 1, wherein the cell culture container is a petri dish or a well plate.

10. The ultrasonic system of claim 1, wherein the temperature sensor is a thermocouple or an infrared sensor.

11. The ultrasonic system of claim 1, wherein the system comprises two or more temperature sensors, whereby the analysis module averages the temperature of the culture medium based on the information provided by the temperature sensors.

12. The ultrasonic system of claim 11, wherein the system comprises at least seven thermocouples.

13. A method of applying low-intensity ultrasound to a cell culture, the method comprising
(i) positioning a cell culture container comprising the cell culture to be in contact with the fluid in the bath of an ultrasonic system of claim 1; and
(ii) applying low-intensity ultrasound to the cell culture.

14. The method of claim 13, wherein the low-intensity ultrasound is applied to the cell culture with a duration of exposure of no more than 60 minutes.

15. The method of claim 13, wherein the cell culture comprises non-adherent cells.

16. The method of claim 13, wherein the cell culture comprises tumor cells, keratinocytes, osteoblasts, osteoclasts, osteocytes, chondrocytes, hepatocytes, islet cells, myocytes, epithelial cells, kidney cells, neurons, stem cells, or a combination thereof.

17. The method of claim 16, wherein the cell culture comprises melanoma cells.

18. A method of inducing apoptosis in a cell, the method comprising
(i) positioning a cell culture container comprising the cell to be in contact with the fluid in the bath of an ultrasonic system of claim 1; and
(ii) applying low-intensity ultrasound to the cell culture.

19.-20. (canceled)

21. The method of claim 18, wherein the cell is a tumor cell, a keratinocyte, an osteoblast, an osteoclast, an osteocyte, a chondrocyte, a hepatocyte, an islet cell, a myocyte, an epithelial cell, a kidney cell, a neuron, or a stem cell.

22. The method of claim 21, wherein the tumor cell is a melanoma cell.
